(19) United States
(12) Patent Application Publication
(10) Pub. No.: US 2025/0241598 A1
(43) Pub. Date: Jul. 31, 2025

(54) ANALYTE SENSOR SENSITIVITY ATTENUATION MITIGATION

(71) Applicant: ABBOTT DIABETES CARE INC., Alameda, CA (US)

(72) Inventors: Gary Alan Hayter, Oakland, CA (US); John Charles Mazza, Long Beach, CA (US); Kenneth J. Doniger, Menlo Park, CA (US)

(73) Assignee: ABBOTT DIABETES CARE INC., Alameda, CA (US)

(21) Appl. No.: 19/182,132

(22) Filed: Apr. 17, 2025

Related U.S. Application Data
(63) Continuation of application No. 16/100,283, filed on Aug. 10, 2018, now Pat. No. 12,285,275.
Application No. 16/100,283 is a continuation of application No. 14/665,603, filed on Mar. 23, 2015, now Pat. No. 10,045,739.
Application No. 14/665,603 is a continuation of application No. 12/242,834, filed on Sep. 30, 2008, now Pat. No. 8,986,208.

(51) Int. Cl. A61B 5/00; A61B 5/145; A61B 5/1468; A61B 5/1486; A61B 5/1495

(52) U.S. Cl. CPC A61B 5/7221; A61B 5/1451; A61B 5/14517; A61B 5/14532; A61B 5/14542; A61B 5/14546; A61B 5/14865; A61B 5/1495; A61B 5/002; A61B 5/1468; A61B 2505/07; A61B 2560/0223; A61B 2560/0481; A61B 2562/043; A61B 2562/046

(57) ABSTRACT

Method and apparatus for receiving a first signal indicative of first glucose levels measured by a first working electrode, receiving a second signal indicative of second glucose levels measured by a second working electrode, detecting a first decrease in an amplitude of the first signal measured by the first working electrode that exceeds a first threshold, detecting a second decrease in an amplitude of the second signal measured by the second working electrode that exceeds the first threshold, selecting the first signal based on determining that the first decrease in the amplitude of the first signal is less than the second decrease in the amplitude of the second signal, determining a rate of change corresponding to the first decrease in the amplitude of the first signal, comparing the rate of change to a second threshold, confirming, based on the comparison, that the first signal is valid.

### RELATED APPLICATIONS

The present application is a continuation of U.S. patent application Ser. No. 16/100,283 filed Aug. 10, 2018, which is a continuation of U.S. patent application Ser. No. 14/665,603 filed Mar. 23, 2015, now U.S. Pat. No. 10,045,739, which is a continuation of U.S. patent application Ser. No. 12/242,834 filed Sep. 30, 2008, now U.S. Pat. No. 8,986,208, entitled “Analyte Sensor Sensitivity Attenuation Mitigation,” the disclosures of each of which are incorporated herein by reference for all purposes.

### BACKGROUND

The detection of the level of analytes, such as glucose, lactate, oxygen, and the like, in certain individuals is vitally important to their health. For example, the monitoring of glucose is particularly important to individuals with diabetes. Diabetics may need to monitor glucose levels to determine when insulin is needed to reduce glucose levels in their bodies or when additional glucose is needed to raise the level of glucose in their bodies.

Accordingly, of interest are devices that allow a user to test for one or more analytes.

### OVERVIEW

In accordance with the various embodiments of the present disclosure, method, device, and system for receiving a first signal from a first working electrode of a glucose sensor positioned at a first predetermined position under the skin layer, receiving a second signal from a second working electrode of the glucose sensor positioned at a second predetermined position under the skin layer, the second signal received substantially contemporaneous to receiving the first signal, detecting a dropout in the signal level associated with one of the first or second signals, comparing the first signal and the second signal to determine a variation between the first and second signals, and confirming one of the first or second signals as a valid glucose sensor signal output when the determined variation between the first and the second signals is less than a predetermined threshold level are provided.

### BRIEF DESCRIPTION OF THE DRAWINGS

FIG. 1 shows a block diagram of an embodiment of a data monitoring and management system according to the present disclosure;

FIG. 2 shows a block diagram of an embodiment of the transmitter unit of the data monitoring and management system of FIG. 1;

FIG. 3 shows a block diagram of an embodiment of the receiver/monitor unit of the data monitoring and management system of FIG. 1;

FIG. 4 shows a schematic diagram of an embodiment of an analyte sensor according to the present disclosure;

FIGS. 5A-5B show a perspective view and a cross sectional view, respectively of another embodiment of an analyte sensor;

FIG. 6 is a graphical illustration of sensor sensitivity attenuation mitigation using multiple working electrodes in accordance with one aspect of the present disclosure;

FIG. 7 is a graphical illustration of sensor sensitivity attenuation mitigation using multiple working electrodes in accordance with another aspect of the present disclosure;

FIGS. 8A-8C are embodiments of analyte sensor electrode geometry for sensitivity attenuation mitigation in accordance with aspects of the present disclosure; and

FIGS. 9A-9B are embodiments of analyte sensor electrode geometry for sensitivity attenuation mitigation in accordance with further aspects of the present disclosure.

### DETAILED DESCRIPTION

Before the present disclosure is described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges as also encompassed within the disclosure, subject to any specifically excluded limit in the stated range. Where the stated range includes one or both of the limits, ranges excluding either or both of those included limits are also included in the disclosure.

It must be noted that as used herein and in the appended claims, the singular forms “a”, “an”, and “the” include plural referents unless the context clearly dictates otherwise.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

The figures shown herein are not necessarily drawn to scale, with some components and features being exaggerated for clarity.

Generally, embodiments of the present disclosure relate to methods and devices for detecting at least one analyte such as glucose in body fluid. Embodiments relate to the continuous and/or automatic in vivo monitoring of the level of one or more analytes using a continuous analyte monitoring system that includes an analyte sensor, at least a portion of which is to be positioned beneath the skin surface of a user for a period of time, and/or the discrete monitoring of one or more analytes using an in vitro blood glucose (“BG”) meter and an analyte test strip. Embodiments include combined or combinable devices, systems and methods and/or transferring data between an in vivo continuous system and a BG meter system.

Accordingly, embodiments include analyte monitoring devices and systems that include an analyte sensor—at least a portion of which is positionable beneath the skin of the user—for the in vivo detection, of an analyte, such as glucose, lactate, and the like, in a body fluid. Embodiments include wholly implantable analyte sensors and analyte sensors in which only a portion of the sensor is positioned under the skin and a portion of the sensor resides above the skin, e.g., for contact to a transmitter, receiver, transceiver, processor, etc. The sensor may be, for example, subcutaneously positionable in a patient for the continuous or periodic monitoring of a level of an analyte in a patient's interstitial fluid. For the purposes of this description, continuous monitoring and periodic monitoring will be used interchangeably, unless noted otherwise. The sensor response may be correlated and/or converted to analyte levels in blood or other fluids. In certain embodiments, an analyte sensor may be positioned in contact with interstitial fluid to detect the level of glucose, in which detected glucose may be used to infer the glucose level in the patient's bloodstream. Analyte sensors may be insertable into a vein, artery, or other portion of the body containing fluid. Embodiments of the analyte sensors of the subject disclosure may be configured for monitoring the level of the analyte over a time period which may range from minutes, hours, days, weeks, or longer.

Of interest are analyte sensors, such as glucose sensors, that are capable of in vivo detection of an analyte for about one hour or more, e.g., about a few hours or more, e.g., about a few days of more, e.g., about three or more days, e.g., about five days or more, e.g., about seven days or more, e.g., about several weeks or at least one month. Future analyte levels may be predicted based on information obtained, e.g., the current analyte level at time to, the rate of change of the analyte, etc. Predictive alarms may notify the user of a predicted analyte level that may be of concern in advance of the user's analyte level reaching the future level. This provides the user an opportunity to take corrective action.

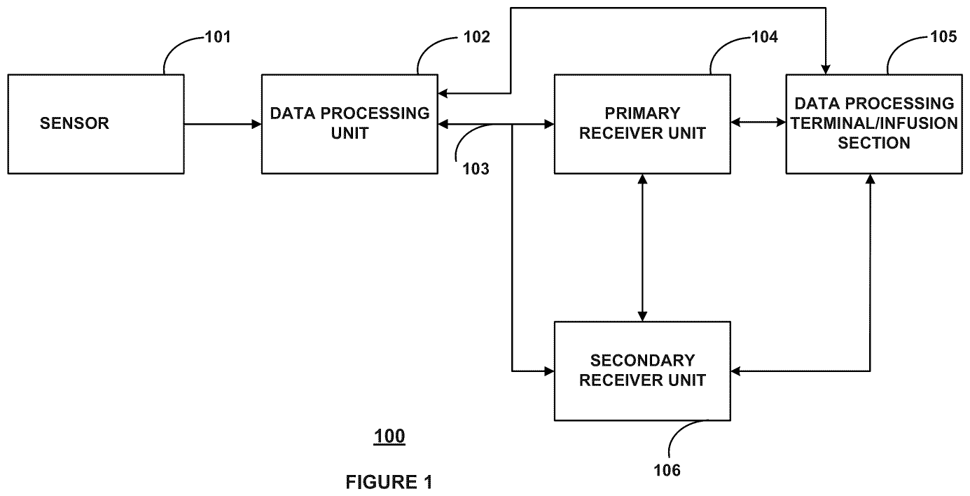

FIG. 1 shows a data monitoring and management system such as, for example, an analyte (e.g., glucose) monitoring system 100 in accordance with certain embodiments. Embodiments of the subject disclosure are further described primarily with respect to glucose monitoring devices and systems, and methods of glucose detection, for convenience only and such description is in no way intended to limit the scope of the disclosure. It is to be understood that the analyte monitoring system may be configured to monitor a variety of analytes at the same time or at different times.

Analytes that may be monitored include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times.

The analyte monitoring system 100 includes a sensor 101, a data processing unit 102 connectable to the sensor 101, and a primary receiver unit 104 which is configured to communicate with the data processing unit 102 via a communication link 103. In certain embodiments, the primary receiver unit 104 may be further configured to transmit data to a data processing terminal 105 to evaluate or otherwise process or format data received by the primary receiver unit 104. The data processing terminal 105 may be configured to receive data directly from the data processing unit 102 via a communication link which may optionally be configured for bi-directional communication. Further, the data processing unit 102 may include a transmitter or a transceiver to transmit and/or receive data to and/or from the primary receiver unit 104 and/or the data processing terminal 105 and/or optionally the secondary receiver unit 106.

Also shown in FIG. 1 is an optional secondary receiver unit 106 which is operatively coupled to the communication link and configured to receive data transmitted from the data processing unit 102. The secondary receiver unit 106 may be configured to communicate with the primary receiver unit 104, as well as the data processing terminal 105. The secondary receiver unit 106 may be configured for bi-directional wireless communication with each of the primary receiver unit 104 and the data processing terminal 105. As discussed in further detail below, in certain embodiments the secondary receiver unit 106 may be a de-featured receiver as compared to the primary receiver, i.e., the secondary receiver may include a limited or minimal number of functions and features as compared with the primary receiver unit 104. As such, the secondary receiver unit 106 may include a smaller (in one or more, including all, dimensions), compact housing or embodied in a device such as a wrist watch, arm band, etc., for example. Alternatively, the secondary receiver unit 106 may be configured with the same or substantially similar functions and features as the primary receiver unit 104. The secondary receiver unit 106 may include a docking portion to be mated with a docking cradle unit for placement by, e.g., the bedside for night time monitoring, and/or a bi-directional communication device. A docking cradle may recharge a power supply.

Only one sensor 101, data processing unit 102, and data processing terminal 105 are shown in the embodiment of the analyte monitoring system 100 illustrated in FIG. 1. However, it will be appreciated by one of ordinary skill in the art that the analyte monitoring system 100 may include more than one sensor 101, and/or more than one data processing unit 102, and/or more than one data processing terminal 105. Multiple sensors may be positioned in a patient for analyte monitoring at the same or different times. In certain embodiments, analyte information obtained by a first positioned sensor may be employed as a comparison to analyte information obtained by a second sensor. This may be useful to confirm or validate analyte information obtained from one or both of the sensors. Such redundancy may be useful if analyte information is contemplated in critical therapy-related decisions. In certain embodiments, a first sensor may be used to calibrate a second sensor.

The analyte monitoring system 100 may be a continuous monitoring system, or semi-continuous, or a discrete monitoring system. In a multi-component environment, each component may be configured to be uniquely identified by one or more of the other components in the system so that communication conflict may be readily resolved between the various components within the analyte monitoring system 100. For example, unique IDs, communication channels, and the like, may be used.

In certain embodiments, the sensor 101 is physically positioned in or on the body of a user whose analyte level is being monitored. The sensor 101 may be configured to at least periodically sample the analyte level of the user and convert the sampled analyte level into a corresponding signal for transmission by the data processing unit 102. The data processing unit 102 is coupleable to the sensor 101 so that both devices are positioned in or on the user's body, with at least a portion of the analyte sensor 101 positioned transcutaneously. The data processing unit may include a fixation element such as adhesive or the like to secure it to the user's body. A mount (not shown) attachable to the user and mateable with the unit 102 may be used. For example, a mount may include an adhesive surface. The data processing unit 102 performs data processing functions, where such functions may include but are not limited to, filtering and encoding of data signals, each of which corresponds to a sampled analyte level of the user, for transmission to the primary receiver unit 104 via the communication link 103. In one embodiment, the sensor 101 or the data processing unit 102 or a combined sensor/data processing unit may be wholly implantable under the skin layer of the user.

In certain embodiments, the primary receiver unit 104 may include an analog interface section including an RF receiver and an antenna that is configured to communicate with the data processing unit 102 via the communication link 103, and a data processing section for processing the received data from the data processing unit 102 such as data decoding, error detection and correction, data clock generation, data bit recovery, etc., or any combination thereof.

In operation, the primary receiver unit 104 in certain embodiments is configured to synchronize with the data processing unit 102 to uniquely identify the data processing unit 102, based on, for example, an identification information of the data processing unit 102, and thereafter, to periodically receive signals transmitted from the data processing unit 102 associated with the monitored analyte levels detected by the sensor 101.

Referring again to FIG. 1, the data processing terminal 105 may include a personal computer, a portable computer such as a laptop, a handheld device (e.g., personal digital assistants (PDAs), telephone such as a cellular phone (e.g., a multimedia and Internet-enabled mobile phone such as an iPhone or similar phone), mp3 player, pager, and the like), or a drug delivery device, each of which may be configured for data communication with the receiver via a wired or a wireless connection. Additionally, the data processing terminal 105 may further be connected to a data network (not shown) for storing, retrieving, updating, and/or analyzing data corresponding to the detected analyte level of the user.

The data processing terminal 105 may include an infusion device such as an insulin infusion pump or the like, which may be configured to administer insulin to patients, and which may be configured to communicate with the primary receiver unit 104 for receiving, among others, the measured analyte level. Alternatively, the primary receiver unit 104 may be configured to integrate an infusion device therein so that the primary receiver unit 104 is configured to administer insulin (or other appropriate drug) therapy to patients, for example, for administering and modifying basal profiles, as well as for determining appropriate boluses for administration based on, among others, the detected analyte levels received from the data processing unit 102. An infusion device may be an external device or an internal device (wholly implantable in a user).

In certain embodiments, the data processing terminal 105, which may include an insulin pump, may be configured to receive the analyte signals from the data processing unit 102, and thus, incorporate the functions of the primary receiver unit 104 including data processing for managing the patient's insulin therapy and analyte monitoring. In certain embodiments, the communication link 103, as well as one or more of the other communication interfaces shown in FIG. 1, may use one or more of: an RF communication protocol, an infrared communication protocol, a Bluetooth® enabled communication protocol, an 802.11x wireless communication protocol, or an equivalent wireless communication protocol which would allow secure, wireless communication of several units (for example, per HIPAA requirements), while avoiding potential data collision and interference.

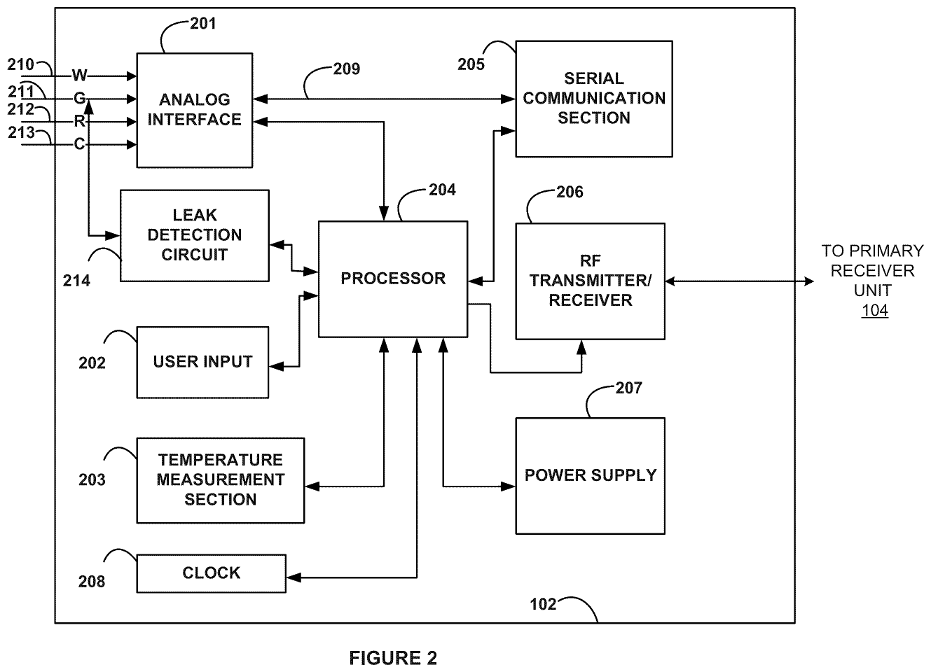

FIG. 2 shows a block diagram of an embodiment of a data processing unit of the data monitoring and detection system shown in FIG. 1. User input and/or interface components may be included or a data processing unit may be free of user input and/or interface components. In certain embodiments, one or more application-specific integrated circuits (ASIC) may be used to implement one or more functions or routines associated with the operations of the data processing unit (and/or receiver unit) using for example one or more state machines and buffers. Referring to the Figure, the data processing unit 102 in one embodiment includes an analog interface 201 configured to communicate with the sensor 101 (FIG. 1), a user input 202, and a temperature measurement section 203, each of which is operatively coupled to a processor 204 such as a central processing unit (CPU).

As can be seen in the embodiment of FIG. 2, the sensor 101 (FIG. 1) includes four contacts, three of which are electrodes-work electrode (W) 210, reference electrode (R) 212, and counter electrode (C) 213, each operatively coupled to the analog interface 201 of the data processing unit 102. This embodiment also shows optional guard contact (G) 211. Fewer or greater electrodes may be employed. For example, the counter and reference electrode functions may be served by a single counter/reference electrode, there may be more than one working electrode and/or reference electrode and/or counter electrode, etc. The electronics of the processing unit and the sensor are operated using a power supply 207, e.g., a battery. Additionally, as can be seen from the Figure, clock 208 is provided to, among others, supply real time information to the processor 204. In one embodiment, a unidirectional input path is established from the sensor 101 (FIG. 1) and/or manufacturing and testing equipment to the analog interface 201 of the data processing unit 102, while a unidirectional output is established from the output of the RF transmitter 206 of the data processing unit 102 for transmission to the primary receiver unit 104. In this manner, a data path is shown in FIG. 2 between the aforementioned unidirectional input and output via a dedicated link 209 from the analog interface 201 to serial communication section 205, thereafter to the processor 204, and then to the RF transmitter 206. Also shown is a leak detection circuit 214 coupled to the guard contact (G) 211 and the processor 204 in the data processing unit 102. The leak detection circuit 214 in accordance with one embodiment of the present invention may be configured to detect leakage current in the sensor 101 to determine whether the measured sensor data are corrupt or whether the measured data from the sensor 101 is accurate.

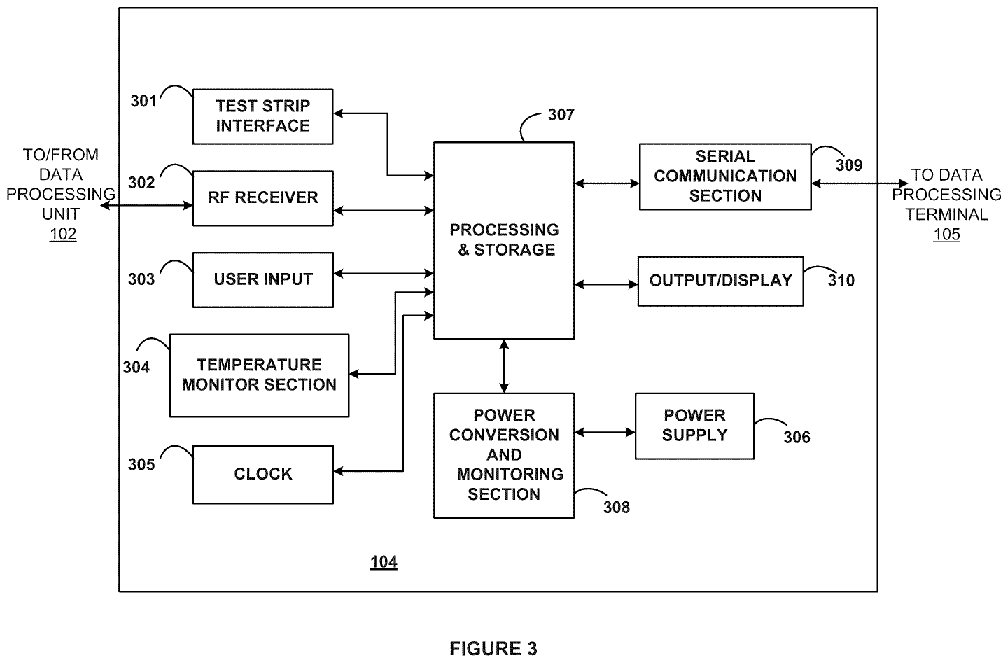

FIG. 3 is a block diagram of an embodiment of a receiver/monitor unit such as the primary receiver unit 104 of the data monitoring and management system shown in FIG. 1. The primary receiver unit 104 includes one or more of: a blood glucose test strip interface 301, an RF receiver 302, an input 303, a temperature detection section 304, and a clock 305, each of which is operatively coupled to a processing and storage section 307. The primary receiver unit 104 also includes a power supply 306 operatively coupled to a power conversion and monitoring section 308. Further, the power conversion and monitoring section 308 is also coupled to the receiver processor 307. Moreover, also shown are a receiver serial communication section 309, and an output 310, each operatively coupled to the processing and storage unit 307. The receiver may include user input and/or interface components or may be free of user input and/or interface components.

In certain embodiments, the test strip interface 301 includes a glucose level testing portion to receive a blood (or other body fluid sample) glucose test or information related thereto. For example, the interface may include a test strip port to receive a glucose test strip. The device may determine the glucose level of the test strip, and optionally display (or otherwise notice) the glucose level on the output 310 of the primary receiver unit 104. Any suitable test strip may be employed, e.g., test strips that only require a very small amount (e.g., one microliter or less, e.g., 0.5 microliter or less, e.g., 0.1 microliter or less) of applied sample to the strip in order to obtain accurate glucose information, e.g. FreeStyle® blood glucose test strips from Abbott Diabetes Care Inc. Glucose information obtained by the in vitro glucose testing device may be used for a variety of purposes, computations, etc. For example, the information may be used to calibrate sensor 101, confirm results of the sensor 101 to increase the confidence thereof (e.g., in instances in which information obtained by sensor 101 is employed in therapy related decisions), etc.

In further embodiments, the data processing unit 102 and/or the primary receiver unit 104 and/or the secondary receiver unit 106, and/or the data processing terminal/infusion section 105 may be configured to receive the blood glucose value wirelessly over a communication link from, for example, a blood glucose meter. In further embodiments, a user manipulating or using the analyte monitoring system 100 (FIG. 1) may manually input the blood glucose value using, for example, a user interface (for example, a keyboard, keypad, voice commands, and the like) incorporated in the one or more of the data processing unit 102, the primary receiver unit 104, secondary receiver unit 106, or the data processing terminal/infusion section 105.

Additional detailed descriptions are provided in U.S. Pat. Nos. 5,262,035; 5,264,104; 5,262,305; 5,320,715; 5,593,852; 6,175,752; 6,650,471; 6,746, 582; 7,299,082, and in application Ser. No. 10/745,878 filed Dec. 26, 2003, now U.S. Pat. No. 7,811,231, entitled “Continuous Glucose Monitoring System and Methods of Use”, each of which is incorporated herein by reference.

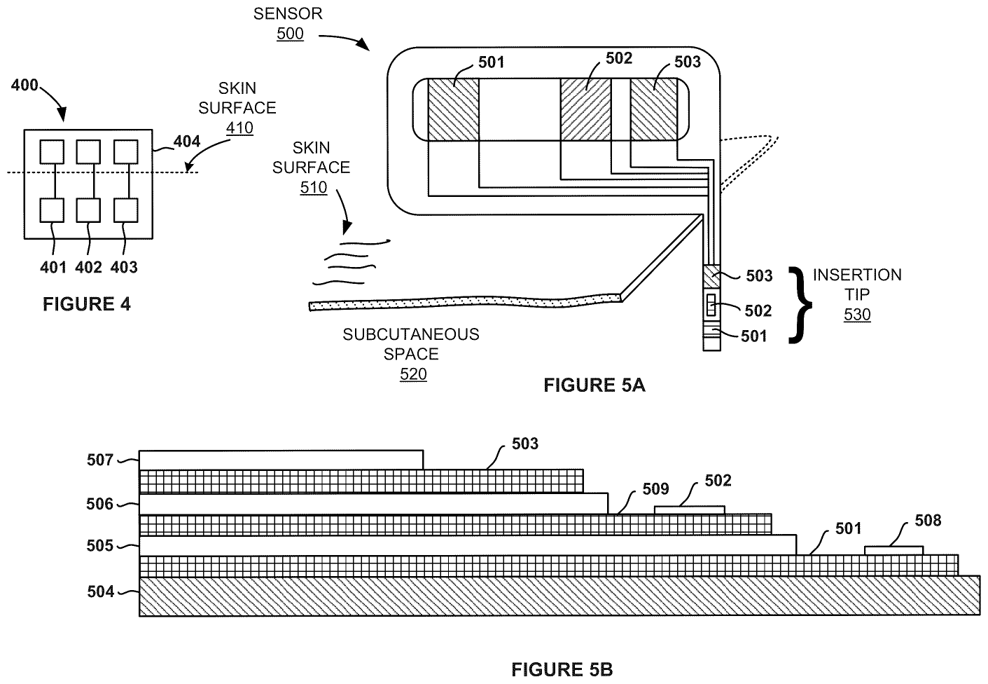

FIG. 4 schematically shows an embodiment of an analyte sensor in accordance with the present disclosure. This sensor embodiment includes electrodes 401, 402 and 403 on a base 404. Electrodes (and/or other features) may be applied or otherwise processed using any suitable technology, e.g., chemical vapor deposition (CVD), physical vapor deposition, sputtering, reactive sputtering, printing, coating, ablating (e.g., laser ablation), painting, dip coating, etching, and the like. Materials include, but are not limited to, aluminum, carbon (such as graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements.

The sensor may be wholly implantable in a user or may be configured so that only a portion is positioned within (internal) a user and another portion outside (external) a user. For example, the sensor 400 may include a portion positionable above the surface of the skin 410, and a portion positioned below the skin. In such embodiments, the external portion may include contacts (connected to respective electrodes of the second portion by traces) to connect to another device also external to the user such as a transmitter unit. While the embodiment of FIG. 4 shows three electrodes side-by-side on the same surface of base 404, other configurations are contemplated, e.g., fewer or greater electrodes, some or all electrodes on different surfaces of the base or present on another base, some or all electrodes stacked together, electrodes of differing materials and dimensions, etc.

FIG. 5A shows a perspective view of an embodiment of an electrochemical analyte sensor 500 having a first portion (which in this embodiment may be characterized as a major portion) positionable above a surface of the skin 510, and a second portion (which in this embodiment may be characterized as a minor portion) that includes an insertion tip 530 positionable below the skin, e.g., penetrating through the skin and into, e.g., the subcutaneous space 520, in contact with the user's biofluid such as interstitial fluid. Contact portions of a working electrode 501, a reference electrode 502, and a counter electrode 503 are positioned on the portion of the sensor 500 situated above the skin surface 510. Working electrode 501, a reference electrode 502, and a counter electrode 503 are shown at the second section and particularly at the insertion tip 530. Traces may be provided from the electrode at the tip to the contact, as shown in FIG. 5A. It is to be understood that greater or fewer electrodes may be provided on a sensor. For example, a sensor may include more than one working electrode and/or the counter and reference electrodes may be a single counter/reference electrode, etc.

FIG. 5B shows a cross sectional view of a portion of the sensor 500 of FIG. 5A. The electrodes 501, 502 and 503, of the sensor 500 as well as the substrate and the dielectric layers are provided in a layered configuration or construction. For example, as shown in FIG. 5B, in one aspect, the sensor 500 (such as the sensor 101 FIG. 1) includes a substrate layer 504 and a first conducting layer 501 such as carbon, gold, etc., disposed on at least a portion of the substrate layer 504 which may provide the working electrode. Also shown disposed on at least a portion of the first conducting layer 501 is a sensing layer 508.

A first insulation layer such as a first dielectric layer 505 is disposed or layered on at least a portion of the first conducting layer 501, and further, a second conducting layer 509 may be disposed or stacked on top of at least a portion of the first insulation layer (or dielectric layer) 505. As shown in FIG. 5B, the second conducting layer 509 may provide the reference electrode 502, and in one aspect, may include a layer of silver/silver chloride (Ag/AgCl), gold, etc.

A second insulation layer 506 such as a dielectric layer in one embodiment may be disposed or layered on at least a portion of the second conducting layer 509. Further, a third conducting layer 503 may provide the counter electrode 503. It may be disposed on at least a portion of the second insulation layer 506. Finally, a third insulation layer 507 may be disposed or layered on at least a portion of the third conducting layer 503. In this manner, the sensor 500 may be layered such that at least a portion of each of the conducting layers is separated by a respective insulation layer (for example, a dielectric layer). The embodiment of FIGS. 5A and 5B show the layers having different lengths. Some or all of the layers may have the same or different lengths and/or widths.

In certain embodiments, some or all of the electrodes 501, 502, 503 may be provided on the same side of the substrate 504 in the layered construction as described above, or alternatively, may be provided in a co-planar manner such that two or more electrodes may be positioned on the same plane (e.g., side-by side (e.g., parallel) or angled relative to each other) on the substrate 504. For example, co-planar electrodes may include a suitable spacing there between and/or include dielectric material or insulation material disposed between the conducting layers/electrodes. Furthermore, in certain embodiments one or more of the electrodes 501, 502, 503 may be disposed on opposing sides of the substrate 504. In such embodiments, contact pads may be on the same or different sides of the substrate. For example, an electrode may be on a first side and its respective contact may be on a second side, e.g., a trace connecting the electrode and the contact may traverse through the substrate.

As noted above, analyte sensors may include an analyte-responsive enzyme to provide a sensing component or sensing layer. Some analytes, such as oxygen, can be directly electrooxidized or electroreduced on a sensor, and more specifically at least on a working electrode of a sensor. Other analytes, such as glucose and lactate, require the presence of at least one electron transfer agent and/or at least one catalyst to facilitate the electrooxidation or electroreduction of the analyte. Catalysts may also be used for those analytes, such as oxygen, that can be directly electrooxidized or electroreduced on the working electrode. For these analytes, each working electrode includes a sensing layer (see for example sensing layer 408 of FIG. 5B) proximate to or on a surface of a working electrode. In many embodiments, a sensing layer is formed near or on only a small portion of at least a working electrode.

The sensing layer includes one or more components designed to facilitate the electrochemical oxidation or reduction of the analyte. The sensing layer may include, for example, a catalyst to catalyze a reaction of the analyte and produce a response at the working electrode, an electron transfer agent to transfer electrons between the analyte and the working electrode (or other component), or both.

A variety of different sensing layer configurations may be used. In certain embodiments, the sensing layer is deposited on the conductive material of a working electrode. The sensing layer may extend beyond the conductive material of the working electrode. In some cases, the sensing layer may also extend over other electrodes, e.g., over the counter electrode and/or reference electrode (or counter/reference is provided).

A sensing layer that is in direct contact with the working electrode may contain an electron transfer agent to transfer electrons directly or indirectly between the analyte and the working electrode, and/or a catalyst to facilitate a reaction of the analyte. For example, a glucose, lactate, or oxygen electrode may be formed having a sensing layer which contains a catalyst, such as glucose oxidase, lactate oxidase, or laccase, respectively, and an electron transfer agent that facilitates the electrooxidation of the glucose, lactate, or oxygen, respectively.

In other embodiments the sensing layer is not deposited directly on the working electrode. Instead, the sensing layer may be spaced apart from the working electrode and separated from the working electrode, e.g., by a separation layer. A separation layer may include one or more membranes or films or a physical distance. In addition to separating the working electrode from the sensing layer, the separation layer may also act as a mass transport limiting layer and/or an interferent eliminating layer and/or a biocompatible layer.

In certain embodiments which include more than one working electrode, one or more of the working electrodes may not have a corresponding sensing layer, or may have a sensing layer which does not contain one or more components (e.g., an electron transfer agent and/or catalyst) needed to electrolyze the analyte. Thus, the signal at this working electrode may correspond to background signal, which may be removed from the analyte signal obtained from one or more other working electrodes that are associated with fully-functional sensing layers by, for example, subtracting the signal.

In certain embodiments, the sensing layer includes one or more electron transfer agents. Electron transfer agents that may be employed are electroreducible and electrooxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes such as ferrocene. Examples of inorganic redox species are hexacyanoferrate (III), ruthenium hexamine, etc.

In certain embodiments, electron transfer agents have structures or charges which prevent or substantially reduce the diffusional loss of the electron transfer agent during the period of time that the sample is being analyzed. For example, electron transfer agents include, but are not limited to, a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments, the redox species is a transition metal compound or complex, e.g., osmium, ruthenium, iron, and cobalt compounds or complexes. It will be recognized that many redox species described for use with a polymeric component may also be used, without a polymeric component.

One type of polymeric electron transfer agent contains a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation. Another example of an ionically-bound mediator is a positively charged polymer such as quaternized poly(4-vinyl pyridine) or poly(1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include, but are not limited to, 4,4′-dimethyl-2,2′-bipyridine and mono-, di-, and polyalkoxy-2,2′-bipyridines, such as 4,4′-dimethoxy-2,2′-bipyridine. Derivatives of 1,10-phenanthroline for complexation with the osmium cation include, but are not limited to, 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include, but are not limited to, polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(1-vinyl imidazole).

Embodiments may employ electron transfer agents having a redox potential ranging from about −200 mV to about +200 mV versus the standard calomel electrode (SCE). The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, such as a glucose oxidase, glucose dehydrogenase (e.g., pyrroloquinoline quinone (PQQ), dependent glucose dehydrogenase, flavine adenine dinucleotide (FAD), or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, such as a glucose oxidase, glucose dehydrogenase (e.g., pyrroloquinoline quinone (PQQ), dependent glucose dehydrogenase or oligosaccharide dehydrogenase, flavine adenine dinucleotide (FAD) dependent glucose dehydrogenase, nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent (which, as described above, may be polymeric). A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

Certain embodiments include a Wired Enzyme™ sensing layer (Abbott Diabetes Care) that works at a gentle oxidizing potential, e.g., a potential of about +40 mV. This sensing layer uses an osmium (Os)-based mediator designed for low potential operation and is stably anchored in a polymeric layer. Accordingly, in certain embodiments the sensing element is a redox active component that includes (1) Osmium-based mediator molecules attached by stable (bidente) ligands anchored to a polymeric backbone, and (2) glucose oxidase enzyme molecules. These two constituents are crosslinked together.

A mass transport limiting layer (not shown), e.g., an analyte flux modulating layer, may be included with the sensor to act as a diffusion-limiting barrier to reduce the rate of mass transport of the analyte, for example, glucose or lactate, into the region around the working electrodes. The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may provide many functions, e.g., biocompatibility and/or interferent-eliminating, etc.

In certain embodiments, a mass transport limiting layer is a membrane composed of crosslinked polymers containing heterocyclic nitrogen groups, such as polymers of polyvinylpyridine and polyvinylimidazole. Embodiments also include membranes that are made of a polyurethane, or polyether urethane, or chemically related material, or membranes that are made of silicone, and the like.

A membrane may be formed by crosslinking in situ a polymer, modified with a zwitterionic moiety, a non-pyridine copolymer component, and optionally another moiety that is cither hydrophilic or hydrophobic, and/or has other desirable properties, in an alcohol-buffer solution. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly(ethylene glycol), hydroxyl or polyhydroxyl modifiers, may be used to enhance the biocompatibility of the polymer or the resulting membrane.

A membrane may be formed in situ by applying an alcohol-buffer solution of a crosslinker and a modified polymer over an enzyme-containing sensing layer and allowing the solution to cure for about one to two days or other appropriate time period. The crosslinker-polymer solution may be applied to the sensing layer by placing a droplet or droplets of the solution on the sensor, by dipping the sensor into the solution, or the like. Generally, the thickness of the membrane is controlled by the concentration of the solution, by the number of droplets of the solution applied, by the number of times the sensor is dipped in the solution, or by any combination of these factors. A membrane applied in this manner may have any combination of the following functions: (1) mass transport limitation, i.e., reduction of the flux of analyte that can reach the sensing layer, (2) biocompatibility enhancement, or (3) interferent reduction.

The electrochemical sensors may employ any suitable measurement technique. For example, may detect current or may employ potentiometry. Techniques may include, but are not limited to, amperometry, coulometry, and voltammetry. In some embodiments, sensing systems may be optical, colorimetric, and the like.

In certain embodiments, the sensing system detects hydrogen peroxide to infer glucose levels. For example, a hydrogen peroxide-detecting sensor may be constructed in which a sensing layer includes an enzyme such as glucose oxides, glucose dehydrogenase, or the like, and is positioned proximate to the working electrode. The sensing layer may be covered by a membrane that is selectively permeable to glucose. Once the glucose passes through the membrane, it is oxidized by the enzyme and reduced glucose oxidase can then be oxidized by reacting with molecular oxygen to produce hydrogen peroxide.

Certain embodiments include a hydrogen peroxide-detecting sensor constructed from a sensing layer prepared by crosslinking two components together, for example: (1) a redox compound such as a redox polymer containing pendent Os polypyridyl complexes with oxidation potentials of about +200 mV versus SCE, and (2) periodate oxidized horseradish peroxidase (HRP). Such a sensor functions in a reductive mode; the working electrode is controlled at a potential negative to that of the Os complex, resulting in mediated reduction of hydrogen peroxide through the HRP catalyst.

In another example, a potentiometric sensor can be constructed as follows. A glucose-sensing layer is constructed by crosslinking together (1) a redox polymer containing pendent Os polypyridyl complexes with oxidation potentials from about −200 mV to +200 mV versus SCE, and (2) glucose oxidase. This sensor can then be used in a potentiometric mode, by exposing the sensor to a glucose containing solution, under conditions of zero current flow, and allowing the ratio of reduced/oxidized Os to reach an equilibrium value. The reduced/oxidized Os ratio varies in a reproducible way with the glucose concentration, and will cause the electrode's potential to vary in a similar way.

A sensor may also include an active agent such as an anticlotting and/or antiglycolytic agent(s) disposed on at least a portion of a sensor that is positioned in a user. An anticlotting agent may reduce or eliminate the clotting of blood or other body fluid around the sensor, particularly after insertion of the sensor. Examples of useful anticlotting agents include heparin and tissue plasminogen activator (TPA), as well as other known anticlotting agents. Embodiments may include an antiglycolytic agent or precursor thereof. Examples of antiglycolytic agents are glyceraldehyde, fluoride ion, and mannose.

Sensors may be configured to require no system calibration or no user calibration. For example, a sensor may be factory calibrated and need not require further calibrating. In certain embodiments, calibration may be required, but may be done without user intervention, i.e., may be automatic. In those embodiments in which calibration by the user is required, the calibration may be according to a predetermined schedule or may be dynamic, i.e., the time for which may be determined by the system on a real-time basis according to various factors, such as, but not limited to, glucose concentration and/or temperature and/or rate of change of glucose, etc.

Calibration may be accomplished using an in vitro test strip (or other reference), e.g., a small sample test strip such as a test strip that requires less than about 1 microliter of sample (for example FreeStyle® blood glucose monitoring test strips from Abbott Diabetes Care Inc.). For example, test strips that require less than about 1 nanoliter of sample may be used. In certain embodiments, a sensor may be calibrated using only one sample of body fluid per calibration event. For example, a user need only lance a body part one time to obtain a sample for a calibration event (e.g., for a test strip), or may lance more than one time within a short period of time if an insufficient volume of sample is firstly obtained. Embodiments include obtaining and using multiple samples of body fluid for a given calibration event, where glucose values of each sample are substantially similar. Data obtained from a given calibration event may be used independently to calibrate or combined with data obtained from previous calibration events, e.g., averaged including weighted averaged, etc., to calibrate. In certain embodiments, a system need only be calibrated once by a user, where recalibration of the system is not required.

Analyte systems may include an optional alarm system that, e.g., based on information from a processor, warns the patient of a potentially detrimental condition of the analyte. For example, if glucose is the analyte, an alarm system may warn a user of conditions such as hypoglycemia and/or hyperglycemia and/or impending hypoglycemia, and/or impending hyperglycemia. An alarm system may be triggered when analyte levels approach, reach, or exceed a threshold value. An alarm system may also, or alternatively, be activated when the rate of change, or acceleration of the rate of change, in analyte level increase or decrease approaches, reaches or exceeds a threshold rate or acceleration. A system may also include system alarms that notify a user of system information such as battery condition, calibration, sensor dislodgment, sensor malfunction, etc. Alarms may be, for example, auditory and/or visual. Other sensory-stimulating alarm systems may be used including alarm systems which heat, cool, vibrate, or produce a mild electrical shock when activated.

The subject disclosure also includes sensors used in sensor-based drug delivery systems. The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, a processing unit such as a transmitter, a receiver/display unit, and a drug administration system. In some cases, some or all components may be integrated in a single unit. A sensor-based drug delivery system may use data from the one or more sensors to provide necessary input for a control algorithm/mechanism to adjust the administration of drugs, e.g., automatically or semi-automatically. As an example, a glucose sensor may be used to control and adjust the administration of insulin from an external or implanted insulin pump.

In certain circumstances, the analyte sensor experiences a sudden drop in sensitivity (defined, for example, as the amount of electrical signal generated by the sensor for every unit of glucose in the interstitial fluid) followed by a signal recovery, generally referred to as signal dropouts. The signal dropouts may be attributed to attenuation of the glucose flux by, for example, occlusion of the working electrode surface of the analyte sensor due to, for example, presence of gas bubbles, contact with tissue (with or without trauma from sensor insertion), contact with cells, or partial pull out or withdrawal of the sensor from the initial position. Generally, such signal dropouts may trigger a false hypoglycemic alarm leading to misinforming the user or the patient of such condition.

Accordingly, in one aspect, the analyte sensor may be configured to mitigate the sensitivity attenuation due to signal dropouts, for example, based on signal processing using multiple working electrodes of the analyte sensor. More specifically, in one aspect, the analyte sensor may be configured with two working electrodes, each with a separate sensing layer, and where the sensing layer of each working electrode is separated by a predetermined distance on the sensor body. For example, in one aspect, the analyte sensor may include a first working electrode with a first sensing layer disposed substantially at the distal tip of the sensor and positioned, for example, in the interstitial fluid of the user.

The analyte sensor may additionally include a second working electrode (either on the same or opposing side of the sensor) with a second sensing layer disposed at a predetermined distance from the first sensing layer of the first working electrode at the distal tip of the analyte sensor. For example, the second sensing layer of the second working electrode of the analyte sensor may be positioned in the dermal layer of the patient, or alternatively, in the interstitial layer but separated from the first sensing layer of the first working electrode by a predetermined distance within the interstitial layer.

In this manner, in one aspect, by providing multiple working electrodes in the analyte sensor each having a separate sensing layer, when signals from one working electrode is attenuated, for example, due to dropout conditions resulting from presence of occluding material around the sensing layer of the electrode, the other working electrode may not experience the same or similar dropout conditions. For example, in the case where the sensor is partially withdrawn or dislodged, the signals from the working electrode positioned away from the distal tip of the sensor may be attenuated due to loss of contact with the interstitial fluid, while the working electrode with the sensing layer positioned substantially at the distal tip of the sensor may be retained, even with the dislodging, in the interstitial fluid, and thus the signal from this working electrode may not experience dropout conditions, and thus attenuation mitigated.

On the other hand, in cases where the distal tip of the sensor is positioned near or within occluding material in the interstitial layer, the signals from the working electrode with the sensing layer at the distal tip of the sensor may experience dropout conditions, while the signals from the working electrode with the sensing layer at a set distance away from the distal tip of the sensor may not be attenuated. In this manner, in one aspect, with multiple working electrodes positioned at different locations within the interstitial fluid and/or other layer under the skin (such as, for example, the dermal layer), the likelihood of sensor sensitivity attenuation occurring at all the working electrodes at the same time may be minimized, and thus, the data processing unit 102 (FIG. 1) and/or the receiver unit 104/106 may be configured to process signals from multiple working electrodes to minimize the sensor signal attenuation or dropout conditions without valid monitored glucose data.

That is, in one aspect, when the signals from one working electrode of the sensor are attenuated, signals from the other working electrode of the sensor may be relied upon to provide the corresponding monitored glucose levels. By physically separating the distance between the two (or more) working electrodes of the sensor and their respective sensing layer, the likelihood of both (or all) working electrodes experiencing the signal dropout conditions at substantially the same time is minimized.

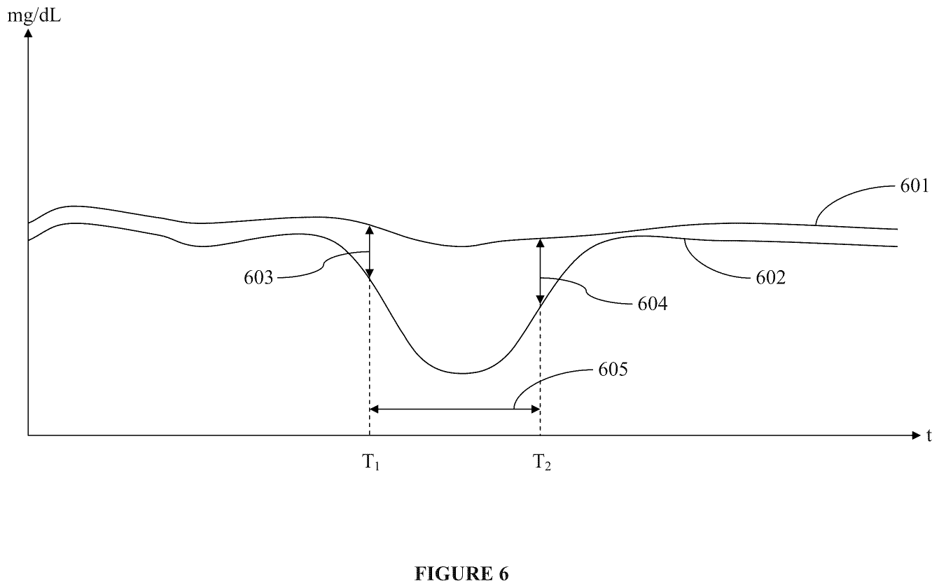

FIG. 6 is a graphical illustration of sensor sensitivity attenuation mitigation using multiple working electrodes in accordance with one aspect of the present disclosure. Referring to FIG. 6, analyte levels monitored from the two working electrodes of the analyte sensor is plotted over a predetermined time period which includes a signal dropout condition. More specifically, signals from the first working electrode 601 and second working electrode 602 are shown. It can be further seen that the signals from the second working electrode 602 has experienced a dropout condition during the time period 605 starting at approximately T1 to T2. During this time period, referring back to FIG. 6, the signals from the first working electrode 601 has not experienced similar signal attenuation.

That is, as shown in FIG. 6, during the time period 605 ranging from T1 and T2, a variation of magnitude between the two electrode signals are substantial. This is illustrated by the indicator 603 and 604 which demonstrate the difference between the analyte levels from the two working electrodes during the time period 605 from T1 and T2. This is in contrast to the difference between the analyte levels from the two working electrodes during the time period preceding time T1 and also during the time period after time T2.

In one aspect, the difference between the signal levels from the two working electrodes are monitored and compared each time they are received (for example, every minute, every 5 minutes, and so on), and a comparison between the signals from the two working electrodes are performed. When the comparison yields a result that returns a difference in the signals from the two working electrodes that exceed a predetermined threshold level (such as, for example, but not limited to, a difference of approximately 15%), in one aspect, the less or smaller signal of the two signals from the respective working electrodes is declared as a dropout signal, and while the other, larger signal from the other of the two working electrodes is considered to be a valid signal. In this manner, referring back to FIG. 6, in one aspect, signals from the second working electrode 602 are considered to be invalid during the time period 605 ranging from time T1 and T2, and the signal from the first working electrode 601 is accepted as the valid signal during this time period 605.

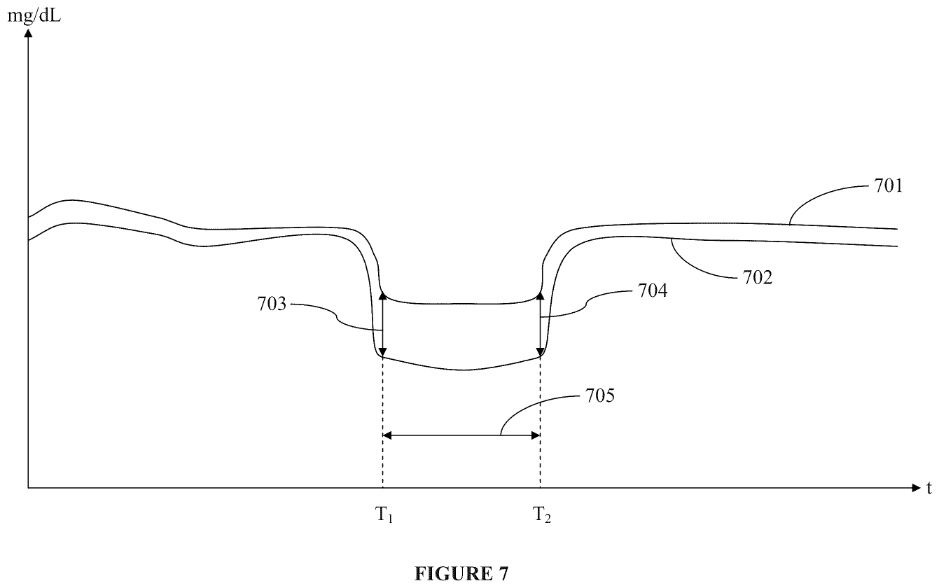

FIG. 7 is a graphical illustration of sensor sensitivity attenuation mitigation using multiple working electrodes in accordance with another aspect of the present disclosure. Referring to FIG. 7, in another aspect, the signals from the two working electrodes may substantially concurrently experience a rapid decrease in the signal level. Indeed, as shown, signals from the first working electrode 701 and the signals from the second working electrode 702 during the time period 705 ranging from time T1 to time T2 illustrate apparent attenuation. Indicator 703 and 704 demonstrate the difference between the analyte levels from the two working electrodes during the time period 705 from T1 and T2. As further shown in FIG. 7, the signals from the first working electrode 701 during this time period 705 are not as attenuated as compared to the signals from the second working electrode 702 during the same time period 705.

Accordingly, when it is detected that signals from both working electrodes appear to experience a dropout type condition or attenuation, in one aspect, signals from both working electrodes may be considered attenuated and invalid. Thus, no valid data or analyte level may be displayed or output to the user during this time period 705. On the other hand, referring again to FIG. 7, when signals from both working electrodes are attenuated, the difference between the magnitude of the signals from the two working electrodes may be determined to confirm attenuation of signals from both working electrodes, or alternatively, to potentially validate one of the two signals from the two working electrodes by determining the rate of change of the signal levels.

That is, in one aspect, when the monitored signals from both working electrodes experience a dropout type condition, for example, as shown substantially at time T1, a rate of change of the signal level is determined based on the signals from the working electrode that is experiencing relatively less dropout (signals from the first working electrode 701 in FIG. 7). The determined rate of change of the signal level in one aspect, is compared to a predetermined threshold level (such as exceeding 0.5 mg/dL/minute, or any other suitable threshold level).

When the rate of the change of the signal level determined does not exceed the predetermined threshold level, in one aspect, the signals from the first working electrode 701 are considered validated and acceptable. That is, even when the signals from the electrodes are undergoing varying degrees of attenuation, by performing a rate of change analysis, it may be determined that the signal attenuation of the lesser of the two signals from the respective working electrodes 701, 702 is attributable to a decline in the corresponding glucose level (rather than an attenuation of the signal itself due to occlusion or some other undesirable condition indicating a false positive potential hypoglycemic condition).

In a further aspect, analysis or processing of signals from two or more working electrodes may also include configurations in which the larger of the two signals at any given time period is considered as valid output signal of the sensor. Alternatively, the signals from the two or more working electrodes may be averaged (for example, when the absolute difference between the two signals is less than a predetermined threshold indicating absence of dropout conditions), weighted based on a predetermined criteria, to determine the corresponding output signal of the analyte sensor.

In another aspect, the shape and/or size of the working electrode may be modified to minimize sensitivity attenuation of the sensor. For example, signal attenuation may result from partial occlusion or blockage of the sensing layer of the sensor working electrode. Accordingly, for an occlusion of a predetermined size, shape and/or location, the resulting signal attenuation may be less for a sensor having a larger working electrode with the sensing layer. Similarly, smaller electrodes may increase the magnitude of the potential signal attenuation, if present, and resulting in easier detection of occlusion or the cause of the signal attenuation.

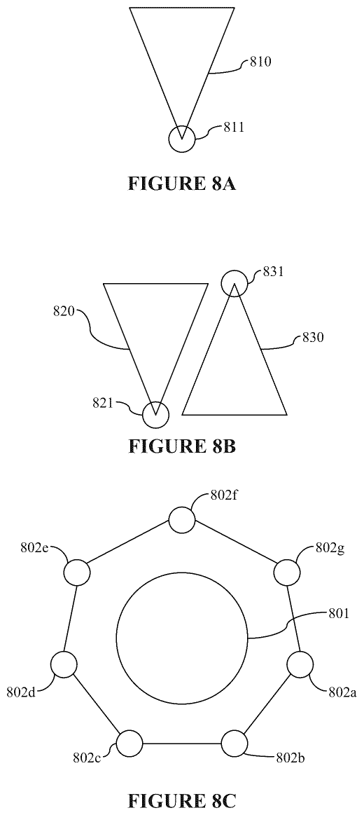

In this manner, in one embodiment, the analyte sensor may be configured to include one relatively large working electrode, and one or more relatively smaller working electrodes that are configured to provide indications that the signals from the relatively large working electrode may be attenuated. By way of an example, FIG. 8C illustrates one embodiment of a sensor working electrode layout including a large working electrode with sensing layer 801 which is surrounded by multiple (in this case, seven) smaller working electrodes 802a, 802b, 802c, 802d, 802c, 802f, and 802g, each of which are electrically connected to each other. When the sensor is positioned with the working electrodes 801 and 802a-802g substantially in fluid contact with the interstitial fluid, presence of occlusion or other signal attenuation condition of the signals from the working electrode 801 may be more easily detected by one or more of the working electrodes 802a-802g, while the larger working electrode 801 may be configured to provide the output sensor signals corresponding to the monitored glucose levels.

Furthermore, in still another aspect, the shape of the working electrode may be configured to minimize signal attenuation or increase the detectability of dropout conditions. For example, as shown in FIG. 8A, the working electrode 810 may be shaped in a triangular shape with the tip portion 811 of the triangular shaped working electrode 810 positioned substantially at the distal end of the analyte sensor. This configuration may minimize signal attenuation by reducing the size of the working electrode at the distal tip of the sensor when occlusions predominantly occur in this area.

FIG. 8B illustrates another embodiment of analyte sensor electrode geometry for sensitivity attenuation mitigation in accordance with aspects of the present disclosure. Referring to FIG. 8B, two working electrodes 820, 830 are shown, each having a substantially triangular shape in the manner shown. In this embodiment, when the occlusion causing signal dropout occurs near the distal end of the sensor where the tip portion 821 of the working electrode 820 is located (that is, for example, the occlusion is slowly moving its way up the sensor from the distal tip), working electrode 820 may be configured to output the sensor signal, while the working electrode 830 may be configured to detect the presence of the occlusion resulting in the signal attenuation. That is, in one aspect, the larger end of the triangular shaped working electrode 830, opposite its tip portion 831, positioned proximate to the tip portion 821 of the working electrode 820, given its size, may detect signal attenuation or occlusion more easily, as compared to the tip portion 821 of the working electrode 820 which is relatively smaller.

In another aspect, in the case where occlusion (such as tissue mass or some blockage) may migrate across the two working electrodes of the analyte sensor, the signals from one working electrode may attenuate before the signals in the other working electrode are attenuated. In this case, a temporal difference between the signals from the two working electrodes exceeding a predetermined threshold may indicate that the analyte sensor signal dropout is occurring. In other words, the signals from the first working electrode that the occlusion edge has come into contact with, migrating over it, may experience signal attenuation before the same or similar degree of signal attenuation is experienced by the second working electrode.

Furthermore, a similar determination may be made upon signal recovery from a dropout, where the recovery occurs at a different time for one working electrode versus the other working electrode. Also, it is to be noted that each electrode may have a different time lag response to abrupt changes in glucose and that the specific lag may be taken into consideration for these comparisons or determinations.

As discussed above, other geometries, shapes, and sizes may be contemplated within the scope of the present disclosure including multiple working electrodes with predetermined positioning of the sensing layer on the working sensor body to mitigate sensor signal attenuation.

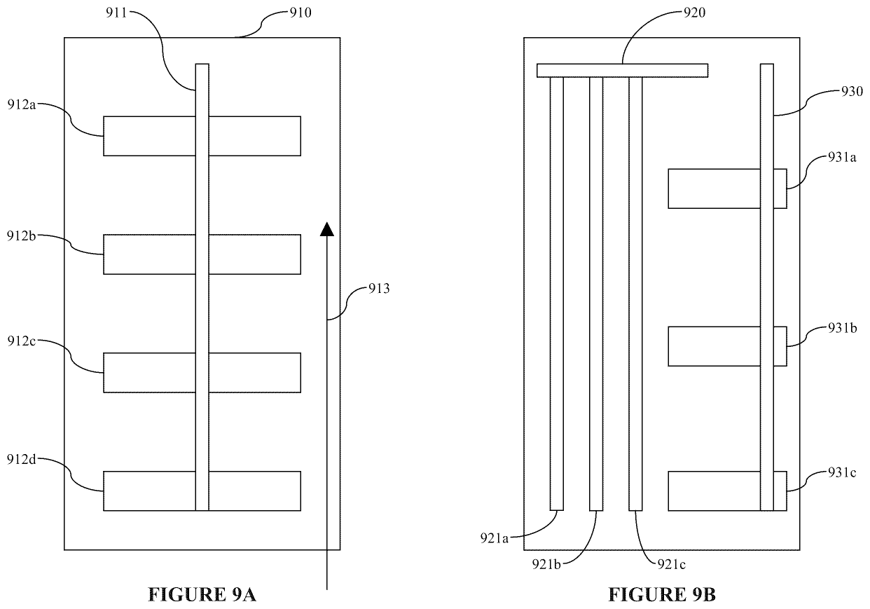

FIGS. 9A-9B illustrate embodiments of analyte sensor electrode geometry for sensitivity attenuation mitigation in accordance with further aspects of the present disclosure. Referring to FIG. 9A, geometry of working electrode 910 is shown to include multiple sensing areas 912a, 912b, 912c, and 912d, which are electrically coupled to the contact or connector 911. Accordingly, when the occlusion edge moves vertically in the direction shown by arrow 913, the sensing areas 912a, 912b, 912c, and 912d will sequentially come into contact with the occlusion edge, and thus the working electrode signals will display sudden changes or drops in sensitivity in, for example, a cascading or in a step wise manner. That is, as the occlusion moves over the working electrode 910 and covers or blocks the sensing areas 912a, 912b, 912c, and 912d, with each sensing area blocked, the corresponding signal from the sensor drops rapidly. Accordingly, signals that display such characteristics or profiles may be associated with actual sensor sensitivity attenuation, rather than a false positive indication of a potential signal dropout.

Referring to FIG. 9B, as shown, in a further aspect, two working electrodes 920, 930 each with multiple sensing areas 921a, 921b, 921c, and 931a, 931b, 931c, respectively, are provided on the analyte sensor with different orientations. As discussed above, an analyte sensor having multiple working electrodes, each with multiple sensing areas, may be configured to form sensitive areas for occlusion detection for identifying signal attenuation. While the embodiments shown and described above in conjunction with the figures illustrate particular number of working electrodes with specific number of sensing areas, as well as orientation, geometry, and position, within the scope of the present disclosure, other configurations including the number of working electrodes, the number of sensing areas for each working electrode, the orientation, geometry, and position of each working electrode and each sensing area are contemplated for analyte sensor.

In one embodiment, a method may comprise, receiving a first signal from a first working electrode of a glucose sensor positioned at a first predetermined position under the skin layer, receiving a second signal from a second working electrode of the glucose sensor positioned at a second predetermined position under the skin layer, the second signal received substantially contemporaneous to receiving the first signal, detecting a dropout in the signal level associated with one of the first or second signals, comparing the first signal and the second signal to determine a variation between the first and second signals, and confirming one of the first or second signals as a valid glucose sensor signal output when the determined variation between the first and the second signals is less than a predetermined threshold level.

In one aspect, the predetermined threshold level may be approximately 15%.

In another aspect, the valid glucose sensor signal may be the larger signal between the first signal and the second signal.

Moreover, the other one of the first or second signals may be confirmed as an invalid signal.

Furthermore, when the variation between the first and the second signals exceeds the predetermined threshold level, confirming both of the first or second signals as invalid sensor output signal.

Furthermore, when the variation between the first and the second signal exceeds the predetermined threshold level, performing a rate of change determination of signals from one of the first or the second working electrodes, and confirming one of the first or the second signals as the valid glucose sensor signal when the determined rate of change level is less than a predetermined threshold level.

Moreover, the predetermined threshold level used to compare the rate of change level may include approximately 0.5 mg/dL per minute.

Moreover, the rate of change determination may be performed on signals from the one of the first or the second working electrodes that is larger in magnitude.

Furthermore, when the determined rate of change level is greater than the predetermined threshold level, confirming both of the first and the second signals from the first and the second working electrodes, respectively, as invalid sensor signals.

In yet another aspect, the first predetermined position may be separated by a predetermined distance from the second predetermined position.

In another aspect, at least a portion of the first working electrode may be in fluid contact with an interstitial fluid, and further, wherein the second working electrode may include at least a portion in fluid contact with the interstitial fluid, and further, wherein the first predetermined position and the second predetermined position may be separated by a predetermined distance.

In yet another aspect, the first predetermined position may include at least a portion of the first working electrode in fluid contact with an interstitial fluid, and further, wherein the second predetermined position may include substantially the entire second working electrode not in fluid contact with the interstitial fluid.

In still another aspect, the first working electrode may be disposed on a first surface of the glucose sensor, and further, wherein the second working electrode may be disposed on a second surface of the glucose sensor.

Furthermore, the first surface and the second surface may be co-planar.

Furthermore still, the first surface and the second surface may be on opposing sides of the glucose sensor.

In one embodiment, an apparatus may comprise a data communication interface, one or more processors coupled to the data communication interface, and a memory storing instructions which, when executed by the one or more processors, receives a first signal from a first working electrode of a glucose sensor positioned at a first predetermined position under the skin layer, receives a second signal from a second working electrode of the glucose sensor positioned at a second predetermined position under the skin layer, the second signal received substantially contemporaneous to receiving the first signal, detects a dropout in the signal level associated with one of the first or second signals, compares the first signal and the second signal to determine a variation between the first and second signals, and confirming one of the first or second signals as a valid glucose sensor signal output when the determined variation between the first and the second signals is less than a predetermined threshold level.

In one aspect, when the variation between the first and the second signals exceeds the predetermined threshold level, the memory storing instructions which, when executed by the one or more processors, may confirm both of the first or second signals as invalid sensor output signals.

In another aspect, when the variation between the first and the second signal exceeds the predetermined threshold level, the memory storing instructions which, when executed by the one or more processors, may perform a rate of change determination of signals from one of the first or the second working electrodes, and may confirm one of the first or the second signals as the valid glucose sensor signal when the determined rate of change level is less than a predetermined threshold level.

Furthermore, when the determined rate of change level is greater than the predetermined threshold level, the memory storing instructions which, when executed by the one or more processors, may confirm both of the first and the second signals from the first and the second working electrodes, respectively as invalid sensor signals.

In one embodiment, an apparatus may comprise means for receiving a first signal from a first working electrode of a glucose sensor positioned at a first predetermined position under the skin layer, means for receiving a second signal from a second working electrode of the glucose sensor positioned at a second predetermined position under the skin layer, the second signal received substantially contemporaneous to receiving the first signal, means for detecting a dropout in the signal level associated with one of the first or second signals, means for comparing the first signal and the second signal to determine a variation between the first and second signals, and means for confirming one of the first or second signals as a valid glucose sensor signal output when the determined variation between the first and the second signals is less than a predetermined threshold level.

Various other modifications and alterations in the structure and method of operation of this present disclosure will be apparent to those skilled in the art without departing from the scope and spirit of the present disclosure. Although the present disclosure has been described in connection with specific preferred embodiments, it should be understood that the present disclosure as claimed should not be unduly limited to such specific embodiments. It is intended that the following claims define the scope of the present disclosure and that structures and methods within the scope of these claims and their equivalents be covered thereby.

What is claimed is:

1. An apparatus for monitoring glucose levels in a subject, comprising:
a glucose sensor comprising a first working electrode and a second working electrode, the first working electrode and the second working electrode being configured to be positioned in contact with a fluid under a skin layer of the subject;
one or more processors operatively coupled to the glucose sensor; and
a memory operatively coupled to the one or more processors and configured to store instructions which, when executed by the one or more processors, cause the one or more processors to:
receive a first signal indicative of a first glucose level measured by the first working electrode;
receive a second signal indicative of a second glucose level measured by the second working electrode;
detect a first decrease in an amplitude of the first signal measured by the first working electrode that exceeds a first threshold;
detect a second decrease in an amplitude of the second signal measured by the second working electrode that exceeds the first threshold;
select the first signal based on determining that the first decrease in the amplitude of the first signal is less than the second decrease in the amplitude of the second signal;
determine a rate of change corresponding to the first decrease in the amplitude of the first signal;
compare the rate of change to a second threshold;
confirm, based on the comparison, that the first signal is valid; and
cause the first signal indicative of the first glucose level to be output to a display or a transmitter.

2. The apparatus of claim 1, wherein the instructions further cause the one or more processors to determine that the second signal indicating the second glucose level is associated with a false positive potential hypoglycemic condition.

3. The apparatus of claim 1, wherein the fluid includes an interstitial fluid.

4. The apparatus of claim 1, wherein the first signal is received from a first sensing area of the glucose sensor comprising the first working electrode and wherein the second signal is received from a second sensing area of the glucose sensor comprising the second working electrode.

5. The apparatus of claim 1, wherein the glucose sensor is factory calibrated and the glucose sensor is configured to require no user calibration.

6. The apparatus of claim 1, wherein at least one of the first working electrode or the second working electrode is a working electrode comprising a glucose-responsive enzyme bonded to a polymer disposed on the at least one of the first working electrode or the second working electrode.

7. The apparatus of claim 6, wherein the glucose-responsive enzyme is chemically bonded to the polymer.

8. The apparatus of claim 6, wherein the at least one of the first working electrode or the second working electrode further comprises a mediator.

9. The apparatus of claim 1, wherein at least one of the first working electrode or the second working electrode is a working electrode comprising a mediator bonded to a polymer disposed on the at least one of the first working electrode or the second working electrode.

10. A system for monitoring glucose levels in a subject, comprising:
a glucose sensor comprising a first working electrode and a second working electrode, the first working electrode and the second working electrode being configured to be positioned in contact with a fluid under a skin layer of the subject;
a data processing device communicatively coupled with the glucose sensor; and
a receiving device communicatively coupled to the data processing device, wherein the receiving device comprises one or more processors and a memory operatively coupled to the one or more processors, the memory storing instructions which, when executed by the one or more processors, cause the one or more processors to:
receive a first signal indicative of a first glucose level measured by the first working electrode;
receive a second signal indicative of a second glucose level measured by the second working electrode;
detect a first decrease in an amplitude of the first signal measured by the first working electrode that exceeds a first threshold;
detect a second decrease in an amplitude of the second signal measured by the second working electrode that exceeds the first threshold;
select the first signal based on determining that the first decrease in the amplitude of the first signal is less than the second decrease in the amplitude of the second signal;
determine a rate of change corresponding to the first decrease in the amplitude of the first signal;
compare the rate of change to a second threshold;
confirm, based on the comparison, that the first signal is valid; and
cause the first signal indicative of the first glucose level to be output to a display or a transmitter.

11. The system of claim 10, wherein the instructions further cause the one or more processors to determine that the second signal indicating the second glucose level is associated with a false positive potential hypoglycemic condition.

12. The system of claim 10, wherein the fluid includes an interstitial fluid.

13. The system of claim 10, wherein the first signal is received from a first sensing area of the glucose sensor comprising the first working electrode and wherein the second signal is received from a second sensing area of the glucose sensor comprising the second working electrode.

14. The system of claim 10, wherein the glucose sensor is factory calibrated and the glucose sensor is configured to require no user calibration.

15. The system of claim 10, wherein at least one of the first working electrode or the plurality of second working electrodes is a working electrode comprising a glucose-responsive enzyme bonded to a polymer disposed on the working electrode.

16. The system of claim 15, wherein the glucose-responsive enzyme is chemically bonded to the polymer.

17. The system of claim 15, wherein the at least one of the first working electrode or the second working electrode further comprises a mediator.

18. The system of claim 10, wherein at least one of the first working electrode or the second working electrode is a working electrode comprising a mediator bonded to a polymer disposed on the at least one of the first working electrode or the second working electrode.

19. An apparatus for monitoring analyte levels in a subject, comprising:
an analyte sensor comprising a first working electrode and a second working electrode, the first working electrode and the second working electrode being configured to be positioned in contact with a fluid under a skin layer of the subject;
one or more processors operatively coupled to the analyte sensor; and
a memory operatively coupled to the one or more processors and configured to store instructions which, when executed by the one or more processors, cause the one or more processors to:
receive a first signal indicative of a first analyte level measured by the first working electrode;
receive a second signal indicative of a second analyte level measured by the second working electrode;
detect a first decrease in an amplitude of the first signal measured by the first working electrode that exceeds a first threshold;
detect a second decrease in an amplitude of the second signal measured by the second working electrode that exceeds the first threshold;
select the first signal based on determining that the first decrease in the amplitude of the first signal is less than the second decrease in the amplitude of the second signal;
determine a rate of change corresponding to the first decrease in the amplitude of the first signal;
compare the rate of change to a second threshold;
confirm, based on the comparison, that the first signal is valid; and
cause the first signal indicative of the first analyte level to be output to a display or a transmitter.

20. The apparatus of claim 19, wherein the analyte sensor is configured to monitor at least two analytes, the at least two analytes including glucose and lactate.
